(19) United States
(12) Patent Application Publication
(10) Pub. No.: US 2026/0007588 A1
(43) Pub. Date: Jan. 8, 2026

(54) BRIGHT NATURAL PIGMENTS – MANUFACTURING PROCESS, COMPOSITION AND COSMETIC APPLICATION

(71) Applicant: Sensient Technologies Asia Pacific Pte Ltd., Singapore (SG)

(72) Inventors: Chin Hao MAH, Singapore (SG); Sophie Hong, Singapore (SG); Handong Zhao, Shanghai (CN); Virginie Boulier, Condecourt (FR); Marianne Peltier, Singapore (SG)

(21) Appl. No.: 19/263,765

(22) Filed: Jul. 9, 2025

Related U.S. Application Data
(63) Continuation of application No. PCT/SG2024/050030, filed on Jan. 12, 2024.
(60) Provisional application No. 63/438,990, filed on Jan. 13, 2023.

(51) Int. Cl. A61K 8/49; A61K 8/26; A61K 8/73; A61Q 1/02

(52) U.S. Cl. CPC A61K 8/498; A61K 8/26; A61K 8/73; A61Q 1/02; A61K 2800/432

(57) ABSTRACT

A composition is disclosed including a natural dye, a bentonite clay substrate, maltodextrin, and an optional pH adjuster. The composition maintains color stability after storage at 28° C. and 68% relative humidity for 30 days. The color stability includes maintaining one or more of an L* value, an a* value, or a b* value, as measured by reflectance visible spectroscopy at one or more wavelengths between 400 nm and 800 nm.

### CROSS-REFERENCE TO RELATED APPLICATION

This application is a bypass continuation of International Application Serial No. PCT/SG2024/050030 (Attorney Docket No. 2121.0121), filed Jan. 12, 2024, entitled “BRIGHT NATURAL PIGMENTS-MANUFACTURING PROCESS. COMPOSITION AND COSMETIC APPLICATION” and published as WO 2024/151222.

International Application Serial No. PCT/SG2024/050030 is related to. claims benefit of U.S. Application Ser. No. 63/438,990 (Attorney Docket No. 2121.0020), filed on January 13. 2023. entitled “BRIGHT NATURAL PIGMENTS—MANUFACTURING PROCESS, COMPOSITION AND COSMETIC APPLICATION”.

Each of the foregoing applications are incorporated by reference in their entireties for all purposes.

### STATEMENT REGARDING FEDERALLY SPONSORED RESEARCH

Not applicable.

### REFERENCE TO A SEQUENCE LISTING

Not applicable.

### FIELD OF THE INVENTION

The disclosed technology is generally directed to compositions including natural dyes. More particularly the technology is directed to compositions including natural dyes for cosmetic applications.

### BACKGROUND OF THE INVENTION

Color is one of the key aspects of marketing cosmetic products. The ability to capture consumer attention through bright, bold, and vibrant shades is a factor contributing to a successful marketing campaign. Consumers around the world are increasingly concerned about the safety of products that they are applying onto their bodies. Hence, there is a rapid growing demand for natural colorants from non-synthetic sources. Natural dyes can have a wide range of shades and can be very bright, but are often subject to instability, for example, towards light, temperature, and pH. Anthocyanins, for example, can exhibit a variety of bright colors ranging from yellow to purple. However, anthocyanins are notoriously unstable, where changes in the molecular structure can lead to undesired, irreversible changes in the color such as deterioration to an undesirable brown pigment. Therefore, there exists a desire to stabilize these natural colorants and develop them with a greater diversity of color shades that are suitable for the food, cosmetics and pharmaceutical industries.

Therefore, there exists a desire to stabilize natural dyes such that the desired color is maintained for an extended period, and any product including the natural dye thus has a longer lifetime.

Existing natural substrates generally have a dull physical appearance, unpleasant odor, and often absorb moisture, deteriorating the performance and shelf life of the cosmetic products. Moreover, the supply of these natural substrates remains low which increases the difficulty of deploying them into application usage. Today, synthetic or mineral substrates are generally preferred for use in cosmetic products that are applied onto the body. There remains a need for compositions of natural colorants and natural substrates that are stable over a wide range of conditions while retaining the original color for an extended amount of time in order to meet the growing consumer demand of bright, stable, non-hydroscopic natural cosmetics with less odor.

### BRIEF SUMMARY OF THE INVENTION

In an aspect, a composition is disclosed herein comprising a natural dye in an amount by weight of between about 5% and about 35%, a bentonite clay substrate in an amount by weight of between about 45% and about 60%, maltodextrin in an amount by weight of between about 10% and about 45%, and an optional pH adjuster, wherein the natural dye is at least partly integrated into a cation interlayer of the bentonite clay substrate by way of a spray drying process. The composition maintains color stability after storage at 28° C, and 68% relative humidity for 30 days, wherein color stability includes maintaining at least about 50%, at least about 60%, at least about 70%, at least about 80%, or at least about 90% of one or more of an L* value, an a* value, or a b* value, as measured by reflectance visible spectroscopy at one or more wavelengths between 400 nm and 800 nm. Over about 50%, over about 60%, over about 70%, over about 80%, or over about 90% of the cations within the cation interlayer are sodium cations (Na⁺), and the bentonite clay substrate has, prior to having the natural dye at least partly integrated into the cation interlayer, a whiteness of at least about 50%. Prior to having the natural dye at least partly integrated into the cation interlayer, the bentonite clay substrate has an initial cation exchange capacity of at least about 75 meq/100 g.

In another aspect, a composition is disclosed herein comprising a natural dye in an amount by weight of between about 20% and about 35%, a kaolin clay substrate in an amount by weight of between about 55% and about 70%, an optional pH adjuster, and an optional surface treatment, wherein the composition maintains color stability after storage at 28° C, and 68% relative humidity for 30 days. The color stability of the composition includes maintaining at least about 50%, at last about 60%, at least about 70%, at last about 80%, or at least about 90% of one or more of an L* value, an a* value, or a b* value, as measured by reflectance visible spectroscopy at one or more wavelengths between 400 nm and 800 nm, wherein the kaolin clay substrate, prior to contacting the natural dye, has a reflectance brightness of at least about 85% as measured by visible reflectance spectroscopy.

In a further aspect, a method of preparing a powder composition is disclosed, the method comprising the following steps: a) homogenizing a natural dye with either: i) a bentonite clay having a whiteness of at least about 50% and a cation exchange capacity (CEC) of at least about 75 meq/100 g; or ii) a kaolin clay with a reflectance brightness of at least about 85%; b) optionally adjusting pH of the slurry by adding a pH adjuster; and c) spray-drying the slurry with an outlet temperature between about 60° C, and about 120° C, to form a powder.

In another aspect, a natural pigment composition is disclosed, the composition comprising a natural dye in an amount by weight of between about 3% and about 40%, a clay substrate in an amount by weight of between about 20% and about 75%, an optional co-substrate in an amount by weight of between about 0) % and about 45%, an optional pH adjuster, and an optional surface treatment in an amount by weight of between about 1 to about 15%, wherein the natural pigment composition is in powder form.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows the effect of pH on the color of an anthocyanin. Form (A) is red and occurs at acidic, low pH conditions. Form (B) is purple occurs at approximately neutral conditions of PH˜6-7. Form (C) is blue-green and occurs at basic conditions, or pH>7.

FIG. 2 is an illustration of the tetrahedral-octahedral-tetrahedral layers of bentonite.

FIG. 3 shows the CIELAB space for color measurement. CIELAB or CIE L*a*b* is a device-independent, 3D color space that enables accurate measurement and comparison of all perceivable colors using three color values.

FIG. 3a shows the components of lipstick formulations used in the test examples.

FIG. 4 is a table showing the colorimetric values and the corresponding physical appearance of the powdered pigment composites using bentonite/montmorillonite as the main substrate.

FIG. 5 is a table showing the colorimetric values and the corresponding physical appearance of the powdered pigment composites using kaolin as the main substrate.

FIG. 6 is a photograph showing the observations of moisture adsorption on a variety of substrates and pigment composites after 30 days at 28° C, and 68% relative humidity.

FIG. 7 shows colorimetric values of red radish compositions using Kunipia-F and ImerCare Opaque as measured at Day 1 (Fresh) and Day 6.

FIG. 8 shows the corresponding physical color of pigment compositions of FIG. 7 measured at Day 1 (Fresh) and Day 6, as measured by the X-rite spectrophotometer.

FIG. 9 is a table of the colorimetric values as measured by the spectrophotometer on the raw material and the purple sweet potato (PSP) pigment composite.

FIG. 10 shows the physical appearance of the powder and lipsticks and after applying on skin for the PSP pigment composite of FIG. 9.

FIG. 11 shows the physical appearance of the powder and lipsticks and after applying on skin for GB composites.

### DETAILED DESCRIPTION OF THE INVENTION

In view of the instability of natural dyes, the present disclosure provides a pigment composition for bright shades of natural dyes encapsulated within natural substrates to enhance stability over time and towards high temperatures and high humidity conditions. This pigment composition provides improved stability of color over a longer period of time as compared to other commercially attainable products, and can also provide at least one of high brightness, reduced odor, reduced moisture adsorption, and better coverage and homogeneity when used in cosmetic applications. According to an aspect of the disclosure, the pigment composition includes natural dyes such as anthocyanins, and a natural substrate that includes bentonite or kaolin.

### Natural Dyes

The pigment composition as disclosed herein includes a natural dye. The natural dye is a colored substance obtained from a natural source. The natural dye can have a color in the visible wavelength range of between 400 nm to 800 nm. These colors can be red, orange, yellow, green, blue, violet, and blends of these colors.

The source of the natural dye can be a plant extract. The plant extract can be isolated from any part of a plant, for example, the roots, leaves, flowers, or stems. Additionally, and alternatively, sources of the natural dye can include fruit extracts or vegetable extracts. The natural dye can be derived from biotechnology such as bio-synthesized, bio-engineered, or biotechnology derived-color molecules.

Natural dyes can be soluble in water. Water soluble natural dyes include anthocyanins. Anthocyanins can be extracted from many types of plants including red radish, black carrot, purple sweet potato, or butterfly pea. More specifically, anthocyanins can include, for example, Pelargonidin. Peonidin, Cyanindin, Malvidin, Delphinidin, and Petunidin which can be harvested from Red Radish (Raphanus sativus L.), Red Sweet Potato (Ipomoea batatas L.) Lam., Black Carrot (Daucus carota L.), Red Cabbage (Brassica oleracea.) Grape (Vitis vinifera), Elderberry (Sambucus nigra), and Strawberry (Fragaroa ananassa). Further examples of water-soluble natural dyes include betaine from beetroot extract, norbixin from annatto seed, and genipin complex as produced from Gardenia jasminoide or genipa americana fruit extract.

Natural oil-soluble dyes can include, for example, beta-carotene extracted from algae or fungi extract, paprika oleoresin extracted from capsicum, bixin extracted from annatto seed and azulene extracted from chamomile flower. Natural dyes can be in solid form, such as a powder, or in liquid form, such as an oil.

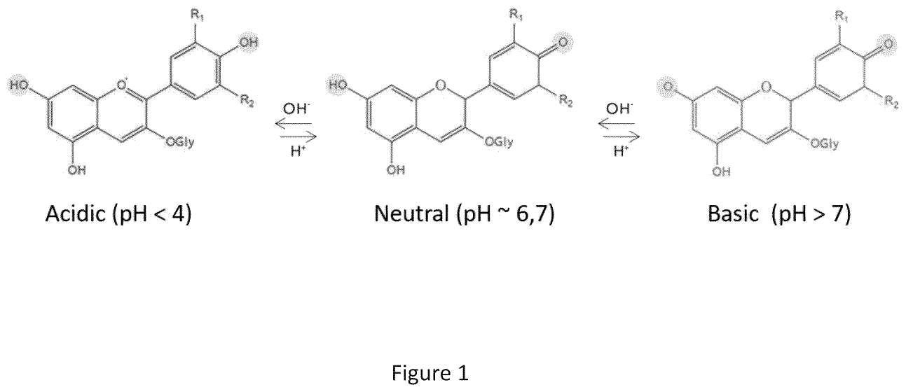

Many natural dyes change color with pH. For example, some anthocyanins color changes from red (acidic) to purple (neutral) to blue-green (basic). FIG. 1 shows the anthocyanin shifting its color from red to green as pH increases. Form (A) is red and occurs at acidic, low pH conditions. Form (B) is purple occurs at approximately neutral conditions of pH ˜6-7. Form (C) is blue-green and occurs at basic conditions, or pH>7.

### Substrate

The pigment composition can include a natural substrate. In certain aspects, the substrate may be a clay, for example bentonite clay, montmorillonite, or kaolin. Bentonite has a layered structure and ion exchange properties that make it ideal for use in cosmetic applications. Bentonite clay has general formula Al₂O₃·4(SiO₂)·H₂O, and is a layered aluminum phyllosilicate mineral. At a molecular level. bentonite clay is structurally arranged such that one layer includes an aluminum octahedral sheet sandwiched between two tetrahedral silica sheets, which is known as a tetrahedral-octahedral-tetrahedral (TOT) structure. The TOT layers are weakly negatively charged and are balanced by exchangeable interlayer cations. The interlayer cations can be sodium (Na⁺), calcium (Ca²⁺) or mixture of these ions. Other ions such as alkali metal ions, alkali earth metal ions, or transition metal ions can be included.

The layers of bentonite can shrink or expand depending on the level of hydration. Each single layer is approximately 1 nm thick. The layers can stack with interlayer cations to several 100 nm when dry. During hydration, the bentonite volume increases significantly. This swelling phenomenon is due to osmotic swelling. The exchangeable cations can diffuse from high concentration areas, such as between the layers, to low concentration of the bulk solution. When the exchangeable cations diffuse out of the lattice, the negatively charged layers experience electrostatic repulsion resulting the volume expansion. This layered molecular structure including interlayer cations provides the substrate with a cation exchange capacity (CEC), or a total capacity of a material to hold exchangeable cations. In an example, the CEC for the bentonite substrate can be at least 75 meq/100 g, optionally at least 80 meq/100 g, or optionally at least 85 meq/100 g.

The cation exchange ability allows alternative ions to exchange with the Na⁺ and or Ca²⁺ while the substrate is in a solution and become encapsulated between the layers when the substrate is dried. For example, when a dry substrate is exposed to a bulk solution of a positively charged natural dye such as that shown in FIG. 1A, the layers move apart and at least some of the cations move out from between the layers. At this point, the positively charged natural dye can replace the exchangeable cations and move in between the layers. The presence of the positively charged natural dyes between the layers stabilizes the negatively charged layers. When the substrate is dried, the positively charged natural dyes can be trapped between the layers. Thus, the natural dye can be at least partially integrated or intercalated within the layers of the substrate.

As a natural substrate, bentonite is available from commercial sources in various forms. As a natural substrate, the present disclosure includes specifically the use of Kunipia-F from Kunimine Industries Co. Ltd. Kunipia is a pure sodium montmorillonite and can have the properties shown in Table 1 below.

TABLE 1
Exemplary properties of Kunipia-F.

| | Kunipia F |
|---|---|
| Viscosity | 200-400 mPa · s |
| Swelling | 45 ml/2 g or more |
| pH | 10 |
| Water content | 10% or less |
| Whiteness | 60 or more |
| Appearance | Fine powder |
| Cation exchange | 115 meq/100 g |

Viscosity: 4%-dispersed solution, BM-type viscometer, 60 rpm25° C.
Whiteness: Brightness by Hunter

Another natural substrate that can be used in the pigment composition as disclosed herein is kaolin which has the formula Al₂Si₂O₅(OH)₄. Kaolin has stacked tetrahedral-octahedral (TO) layers where the T and O layers are composed of silicon and aluminum as described above for bentonite. Kaolin has two sheets in each layer that are strongly bonded together via shared oxygen ions. The layers are bonded to each other via hydrogen bonding between oxygen on the outer face of the T sheet of one layer and hydroxyl group (—OH) on the outer face of the O sheet of the next layer. The kaolin layer has no net electrical charge and so there are no cations (such as calcium, sodium, or potassium) between layers as with bentonite. Accordingly, kaolin has a low ion exchange capacity. Hydrogen bonding between layers also hinders water molecules from infiltrating between layers, accounting for the lack of swelling behavior observed for kaolin.

When moistened, water molecules loosely bond with the platelets of kaolin in such a way that allows the platelets to slip past each other when the clay is manipulated, but also allows the kaolin clay to retain a molded shape. When kaolin is dry, the plates hydrogen bond directly to each other.

Natural substrates can include, but are not limited to. Kunipia-F (Kunimine), ImerCare Opaque (Imerys S.A.), Moistnite-S (Kunimine), Moistnite-U (Kunimine), Caolin G30M (Veneta Mineraria S.P.A), Cosmetico. AV-Beige, AV-Red, AV-Grey, and the like. Additionally, and alternatively, the substrate can be Tricalcium Citrate. TriCalcium Phosphate, Palm seed powder, Silica, Polyhydroxybutyrate, or Diatomite.

As a natural substrate, kaolin is available from commercial sources in various forms. The present disclosure includes specifically the use of kaolin in the form of ImerCare Opaque from Imerys S.A, to achieve the desired performance.

### Whiteness and Brightness Reflectance

Whiteness and brightness reflectance of a substrate are important for cosmetic applications and can be measured using spectrophotometry. Brightness reflectance measures the visible reflectance of for example. 457 nm wavelength of light, whereas whiteness measures the reflection of all wavelengths. The pigment composition according to an aspect disclosed herein can include a substrate having a whiteness of at least 50%, or at least 60%, or at least 70%. Kunipia, the sodium bentonite. for example can have a whiteness of at least 50%, or at least 60%, or at least 70%. The pigment composition can include kaolin having a high brightness reflectance, for example, a brightness reflectance of at least 85%, or at least 90%, or at least 95%.

### Co-Substrate

A co-substrate can be used to add additional cosmetic application properties such as increasing the slip effect for example. Co-substrates can include polysaccharides, for instance maltodextrin, chitosan, Arabic gum, cellulose, pullulan, and the like. Co-substrates can include fillers such as mica, talc, silica, calcium carbonate, and the like. Co-substrates can include mineral pigments such as boron nitride or clays and the like, as well as combinations of polysaccharides, fillers, and mineral pigments.

### pH Adjustment

The color of the natural dye can be adjusted to the desired shade by changing the pH of a solution of the natural dye or of a slurry of the natural dye mixed with the substrate. The pH can be adjusted with an acid or base commonly used in the chemistry field to obtain the right shade or the increase solubility of the natural dyes. According to an aspect of the disclosure herein, citric acid and sodium hydroxide are preferably used. Additionally, and alternatively, acids such as ascorbic acid, an alpha hydroxy acid, a beta hydroxy acid, a volatile acid, or a fatty acid, or a combination thereof can be used. Bases used to adjust the pH can include amines such as organic amines, ammonium hydroxide, sodium hydroxide, potassium hydroxide, or combinations thereof. The pH adjuster can be present in an amount by weight of between 8% and 15%, between 9% and 14%, between 12% and 15%, or between 10% and 12%

### Method of Preparing the Pigment Composition

The method of preparing the pigment composition can include the following steps: (i) homogenization of the natural dye and substrate in a solvent to make a slurry, including pH adjustment as needed to achieve desired color shade or solubility of the natural dye, and (ii) spray-drying of the slurry with outlet temperature from about 60° C, to about 120° C, or between 90° C, and 120° C, between 70° C, and 100° C, or between 60° C, and 90° C.

The solvent can be water, or a volatile organic solvent such as ethanol, or its blends. The solvent can be an emulsion base, for example an oil in water emulsion base. The choice of the solvent will depend on the nature of natural dyes. Water is preferably used for water soluble natural dyes, while the volatile organic solvent or emulsion base pigment compositions can be used for natural oil soluble dyes.

The color shade and/or solubility of the natural dye can be tuned by adjusting the pH of the solution. As shown in FIG. 1, the molecular structure of natural dyes can change according to pH which in turn corresponds to a change in the color and/or the solubility of the natural dye. The pH can be adjusted using an acid or base as described above. The pH of a solution of the natural dye can be adjusted before the addition of the substrate. The pH of a mixture of the substrate and natural dye can be adjusted after the natural dye and substrate are combined with solvent to form a homogeneous suspension or slurry.

The solution or slurry of natural dye combined with the substrate can be spray-dried. An atomizer or spray nozzle disperses the liquid or slurry into a controlled drop size spray. A heated drying gas, such as air, oxygen, nitrogen, or other inert gas, can be passed in the same direction as the sprayed liquid. Alternatively, the gas can flow counter to the liquid flow, where the hot air flows against the flow from the atomizer. After spray-drying, the moisture content is from about 0 wt % to about 10 wt %, between 1% and 9%, between 2% and 5%, and preferably from about 0 wt % to about 5 wt %. This drying procedure effectively lowers the moisture content of the pigment composition. The dried pigment composition can be stabilized against absorbing atmospheric humidity.

Spray-drying of the slurry of natural dye combined with the substrate results in particles of relatively uniform size. The method can further include the optional step of (iii) particle size reduction to prepare the powdered products. The particle size can be reduced and homogenized by grinding, milling, or pulverizing, using a grinder or jet mill. The particle size of the pigment composition in powder form can be from about 1 μm to about 20 μm, preferably about 2 μm to about 10 μm as measured by laser diffraction. Particle size may be between 1 μm to 20 μm, 2 μm to 20 μm, 1 μm to 5 μm, between 5 μm to 20 μm, between 10 μm to 15 μm, or between 5 μm to 15 μm.

### Surface Treatment

The method can further include the optional step (iv) addition of a surface treatment. The surface treatment can additionally be used in post-process according to an aspect of the disclosure. For example, a synthetic or natural surface treatment such as oil, butter, or wax can be used to make natural pigments more hydrophobic and/or lipophilic and improve the dispersion of the natural pigment in oil or silicone media. The surface treatment can include a stearate, such as polyglyceryl-10-pentaisostearate or polyglyceryl-10 nonaisostearate, a wax, such as Meadowfoam wax, an oil or butter, such as Moringa oil or butter or aloe oil or butter, and combinations thereof.

The pigment composition obtained in powder form according to an aspect of this disclosure can be used in cosmetic applications. The pigment composition can be used in powder form such as compact powder. The pigment composition can be used in dispersions such as lipsticks or liquids, such as in a liquid foundation. In these cases, the pigment composition is generally dispersed in an oil, wax, or silicone media. The pigment composition can be dispersed in water in an oil, wax, or silicone emulsion, preferably in a cosmetic formula. The pigment composition can be dispersed in a liquid (such as oil or silicone) or in a solid (such as butter or wax). The pigment composition as disclosed herein can increase brightness of cosmetic pigment composition and brightness on the body (skin, lips, hair, eyelashes) after application.

Cosmetic application in this invention can include, for example, lipsticks, lip-gloss, face make-up products, hair color products, sun care products, mascara, and foundation. Cosmetic formulas can be in powder form, in monophasic or biphasic lotion, water in oil or oil in water emulsion, gel, or cream.

In certain aspects, the pigment composition can include a natural dye in an amount by weight of between about 3% and about 40%, between about 5% and 30%, between about 5% and about 35%, or between about 10% and about 25%. In certain aspects, the pigment composition can include a bentonite clay substrate in an amount by weight of between about 25% and about 75%, between about 50% and about 70%, between about 45% and about 50%, between about 50% and about 65%, or between about 45% and about 60%. In certain aspects, the pigment composition can include a kaolin clay substrate in an amount by weight of between about 45% and about 80%; between about 50% and about 75%, between about 45% and about 50%, between about 50% and about 65%, or between about 55% and about 70%. In certain aspects, the pigment composition can include a co-substrate such as maltodextrin in an amount by weight of between about 1% and about 70%, between about 5% and about 60%, between about 10% and about 30%, between about 20% and about 40%, or between about 10% and about 45%.

In certain aspects, the pigment composition maintains color stability after storage at 28° C. and 68% relative humidity for 30 days, wherein color stability includes maintaining at least about 50%, at last about 60%, at least about 70%, at last about 80%, or at least about 90% of one or more of an L* value, an a* value, or a b* value, as measured by reflectance visible spectroscopy at one or more wavelengths between 400 nm and 800 nm.

As illustrated in the examples below, pigment compositions that include either Kunipia-F or ImerCare Opaque and are prepared according to the method disclosed herein demonstrate exceptional color stability over time and when stored at 28° C, and 68% relative humidity. Factors contributing to this exceptional performance and stability of the compositions having Kunipia-F or ImerCare Opaque materials include the purity, composition, and ion exchange capacity (in the case of Kunipia-F), and the spray-drying method of encapsulating the natural dyes.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

### EXAMPLES

### Methods

### Physical Evaluation

Physical evaluation of the example pigment compositions was observed by humans using the senses of touch, sight, and smell by a person skilled in the cosmetic field. These evaluations include physical observation of odor, homogeneity or coverage, and brightness after application of the pigment composition on skin.

### Humidity Evaluation

High humidity absorption over time will turn natural pigments darker and harder over time. The bentonite, montmorillonite, and kaolin pigment composites were further subjected to moisture adsorption investigation for 30 days using high humidity condition at temperature 28° C, and relative humidity at 68%. This condition was set to mimic the high humidity atmosphere in Asian countries and at the same time illustrating the hygroscopicity of the pigment composites.

### Colorimetric Evaluation

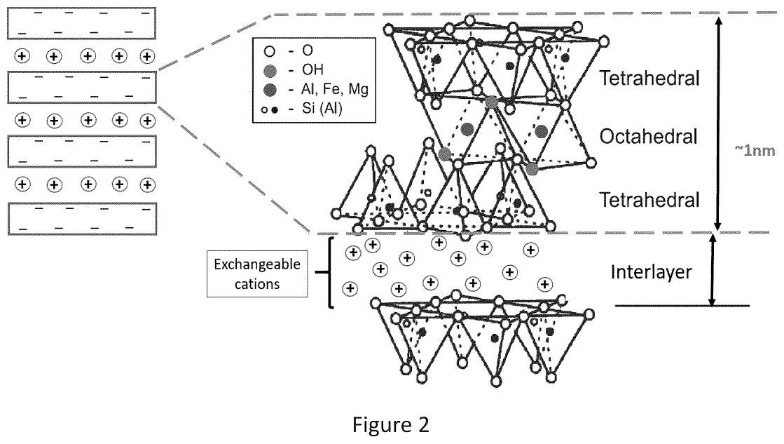

The colorimetric analysis was carried out using X-rite spectrophotometer VS450 with Color iQC programme. The main objective in this colorimetric investigation is to identify which pigment composites give the warmest and brightest shade of red indicating the highest L*, a* and b* values. FIG. 2 shows the CIELAB color sphere which helps to illustrate and select the best pigment composite. The investigation uses commercially available NATPURE XFINE RADISH RR319 (INCI: Raphanus sativus L extract) as the benchmark for comparison purposes.

### Lipstick Formula

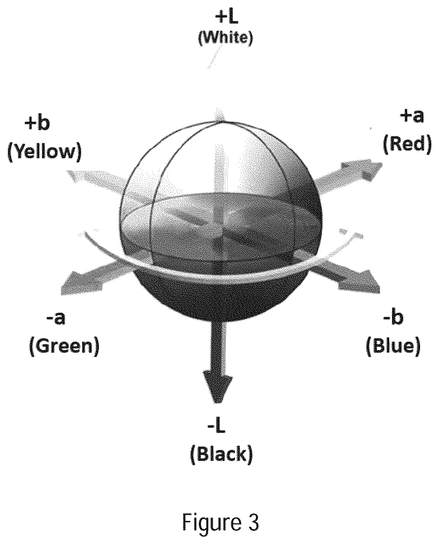

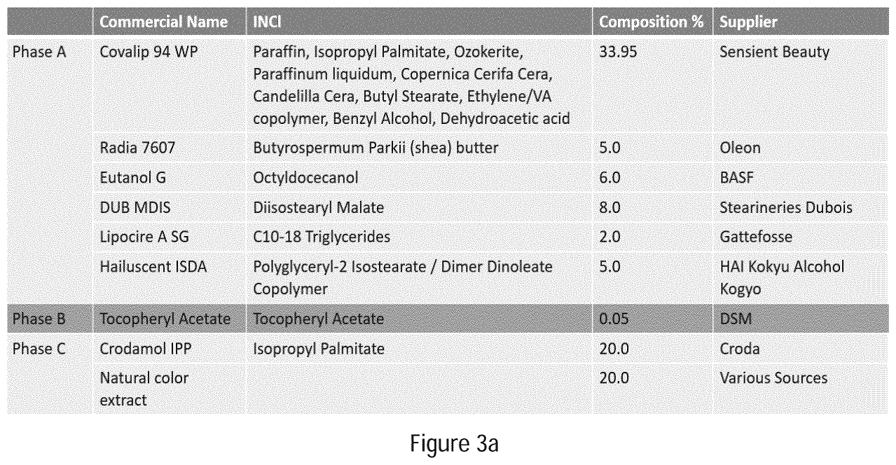

The pigment compositions were tested in some examples as lipsticks. The formulation for the pigment compositions as lipsticks is shown in the table of FIG. 3.

Examples 1 through 5 use the natural water-soluble dye anthocyanins from Raphanus sativus L., referred to as Red Radish (RR). Example 6 uses another source of anthocyanins from Ipomoea batatas L. referred to as Purple Sweet Potato (PSP). Example 7 uses Genipin-hydrolyzed Gardenia jasminoides ellis referred to as Gardenia Blue (GB). The general composition can include natural dyes (1-70 wt %), substrate (30-99 wt %), optional co-substrate (0-60 wt %), optional pH adjuster, and optional surface treatment (1-15 wt %).

### Example 1 Choice of Natural Substrates

The Raphanus sativus L extract powder as the source of anthocyanins was obtained from Sensient Beauty as “NATPURE XFINE RADISH RR319 (INCI: Raphanus sativus L extract)”.

Composition of natural pigments are as follows: Natural dyes: 3-40 wt %; Natural substrates: 20-75 wt %: Option 1—Co-substrate: 0-40 wt %; Option 2—pH adjustment depending on natural dyes with acid or base such as citric acid or sodium hydroxide: Option 3—Surface treatment: 1 to 15 wt %.

The new substrates have been chosen for its opacity appearance: (1) TriCalcium Citrate, (2) TriCalcium Phosphate, (3) Kaolin, (4) Palm seed powder, (5) Montmorillonite, (6) Silica, (7) Polyhydroxy butyrate. (8) Bentonite, and (9) Diatomite.

The newly spray-dried pigment composites undergo evaluation of the following five factors and the results are summarized in Table 2: (A) Brightness in powder form, (B) odor in powder form, (C) moisture content of powder, (D) homogeneity/coverage in lipstick and (E) brightness after application on skin.

Values are given between 0 and 10. A value of 0 corresponds to the case where the natural pigment is not bright, has strong odor, high moisture content, and is not homogenous in coverage in lipstick application and not bright after application on skin. A value of 10 corresponds to the case where the natural pigment is very bright, has no odor, no moisture content, and is homogenous and shows good coverage in lipstick application and very bright after application on skin.

The results shown in Table 2 show that only the kaolin and bentonite lead to bright natural pigments, have low moisture content, little to or no odor, and are homogenous, providing good coverage in the application.

TABLE 2
Summary of the outcome of the various natural
pigments undergoing the five evaluation factors

| | Bright- | | | Homoge- | Brightness |
|---|---|---|---|---|---|
| | ness | Odor | Mois- | neity/ | after |
| | in | in | ture | coverage in | appli- |
| | powder | powder | con- | application | cation |
| Criteria/substrates | form | form | tent | (lipstick) | on skin |
| RR319 (Standard) | 1 | 1 | 0 | 8 | 4 |
| Tricalcium citrate | 5 | 4 | 3 | 5 | 6 |
| Tricalcium phos- | 5 | 4 | 3 | 5 | 5 |
| phate | | | | | |
| Kaolin | 9 | 9 | 9 | 9 | 8 |
| Palm seed powder | 6 | 2 | 2 | 5 | 5 |
| Montmorillonite | 6 | 7 | 4 | 8 | 7 |
| (grey/beige/red) | | | | | |
| Silica | 2 | 2 | 2 | 5 | 6 |
| Polyhydroxybu- | 2 | 2 | 2 | 4 | 5 |
| tyrate | | | | | |
| Bentonite | 10 | 8 | 8 | 9 | 10 |
| Diatomite | 5 | 4 | 4 | 5 | 5 |

### Example 2: Red Radish Extract Encapsulated on Clays: Bentonite vs Montmorillonite

For comparison in a lipstick application, 7 different grades of clay; 4 Bentonites (Kunipia-F, Moistnite-S, Moistnite-U and COSMETICO) and 3 Montmorillonites (grey, beige and red) are used.

These natural pigments comprise from about 20 wt % to about 35 wt % of Xfine Radish RR319, from about 45 wt % to about 60 wt % of bentonite or montmorillonite, from about 10 wt % to about 25 wt % of maltodextrin, and from about 8 wt % to about 15 wt % of citric acid. The citric acid is added to serve as pH adjustment ranging from 2.9 to 3.4 but preferably at 3.0. The commercially available natural clays; bentonite clay (Kunipia-F, Moistnite-U and S) were obtained from Kunimine Industries Co. Ltd., bentonite clay (COSMOTICO) was obtained from SwellWell MineChem Pvt. Ltd., montmorillonite clay (ArgileVerte Grey/Beige/Red) were obtained from Argiles Du Bassin Mediterraneen ABM.

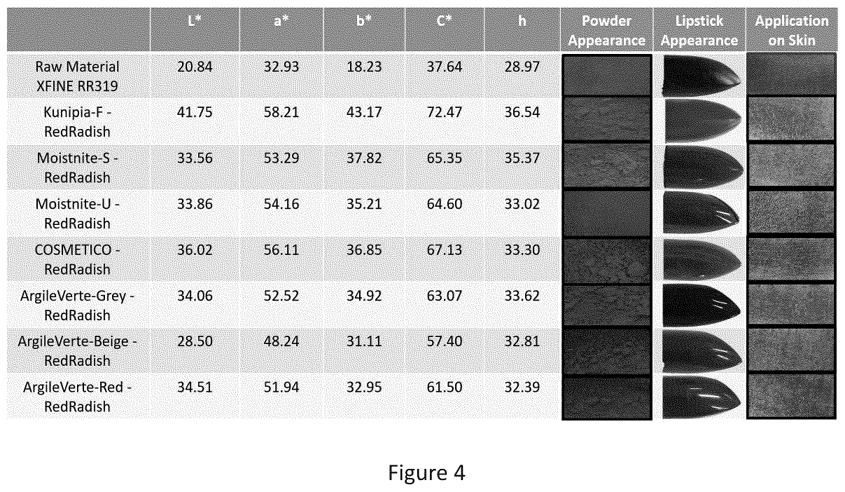

FIG. 4 presents the L*, a*, b*, C* and h results of the powdered pigment composites for Example 2 compositions. The spray-dried ingredients were subsequently subjected to lipstick application testing. Images of the laboratory-scale lipsticks and their corresponding application on skin were recorded and also tabulated in FIG. 4.

From the summary in FIG. 4, the results demonstrate that the Kunipia-F-RedRadish composite has notably exhibited the highest L*, a* and C* values indicating the brightest and reddest among the selected bentonite and montmorillonite. It is also worth noting that the Kunipa-F also provides the warmest shade of red giving the highest b* value. The analytically analyzed values also correspond to the observed brightest physical appearance as lipstick and as applied onto the skin.

### Example 3: Red Radish Extract Encapsulated on Kaolin

For comparison in a lipstick application, 3 different grades of kaolin are used. The Raphanus sativus L extract powder as the source of anthocyanins was obtained from Sensient Beauty as “Red Radish Extract (INCI: Raphanus sativus L, extract)”.

The pigment composite comprises from about 20 wt % to about 35 wt % of Red Radish extract, from about 55 wt % to about 70 wt % of kaolin, and from about 8 wt % to about 15 wt % of citric acid. The natural clay kaolin (Caolin G30M) was obtained from Veneta Mineraria S.P.A and kaolin (ImerCare Opaque) was obtained from Imerys S.A. The composition in this example undergoes surface treatment with about 1 wt % to about 6 wt % of polyglyceryl-10 pentaisotearate. The surface treatment agent is not limited to polyglyceryl-10 pentaisotearate but could be extended with other agents such as Meadowfoam wax, polyglyceryl-10 nonaisotearate, moringa oil/moringa butter, and aloe oil or aloe butter.

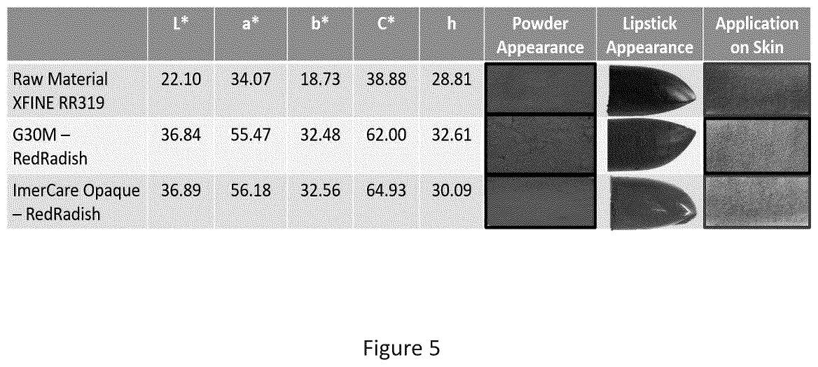

For this embodiment comprising kaolin as the main substrate, the analyzed colorimetric values were recorded and tabulated in FIG. 5. The colorimetric results indicate that the ImerCare Opaque-RedRadish composite can be another effective candidate that improves the brightness and chroma of the pigment. Comparing FIG. 4 and FIG. 5, the difference between the ImerCare Opaque and Kunipia-F pigmented composite is the b* value where the pigmented-bentonite composition showing the highest b* value which gives a warmer (yellow) tone and pigmented-kaolin composition giving a cooler (blue) tone.

### Example 4: Humidity Absorption Evaluation

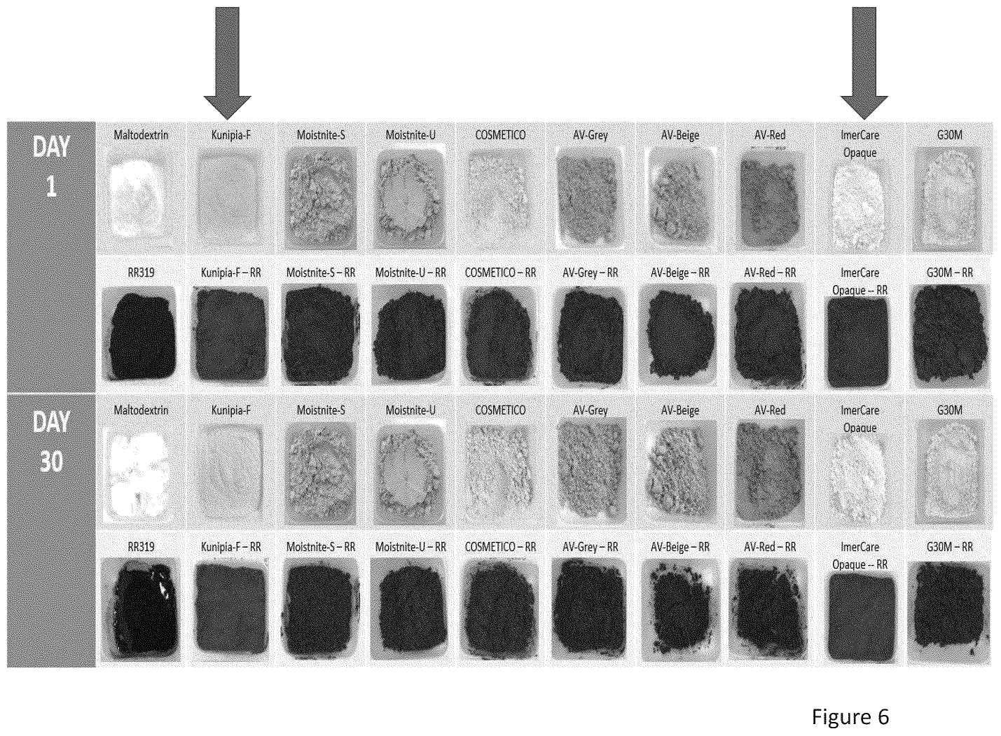

The bentonite, montmorillonite and kaolin pigment composites were further subjected to moisture adsorption investigation for 30 days using high humidity condition at temperature 28° C. and relative humidity at 68%. This condition was set to mimic the high humidity atmosphere in Asian countries and at the same time illustrating the hygroscopicity of the pigment composites. These pigment composites were compared against the benchmark Xfine RR319 and the results were tabulated in FIG. 6.

High humidity absorption over time will turn natural pigments darker and harder over time. Based on the observation shown in FIG. 6, the benchmark Xfine RR319 has turned from maroon powdery state to a black solid state. This observed phenomenon is expected due to the hygroscopic natural of maltodextrin and natural juice extract. The addition of bentonite, montmorillonite, or kaolin in forming the new pigment composites, reduces the moisture adsorption process. However, the retardation of the moisture adsorption is limited to 5 days as hardening and darkening observation are seen on the pigment composites: AV-Beige-RR, AV-Red-RR and G30M-RR. The hardening and darkening of the appearance clearly imply that moisture adsorption has occurred. The observation continues until day 8 where again hardening and darkening appearance is shown on Moistnite-S-RR, Moistnite-U-RR, Cosmetico-RR and AV-Grey-RR. The moisture adsorption investigation ended in day 30 with Kunipia-F-RR and ImerCare Opaque-RR (indicated by arrows) showing neither hardening nor darkening in appearance.

Overall, the colorimetric and humidity evaluation on Raphanus sativus with various co-substrates shows the pigment composite comprising Kunipia-F has exhibited highest L*, a* and C* values indicating the brightest and reddest. The pigment composite ImerCare Opaque-RR is included for further evaluation due to best performance shown in the humidity test.

### Example 5: Color Evolution Evaluation in Lipstick

It was reported that the commercially available Natpure Xfine RR319 experienced color changing effect even it was formulated into lipstick application. The blue-shifting of color on Xfine RR319 was observed after 6 hours of application on skin. Due to the fact that the pH on skin varies, the investigation on the color evolution of the new pigment composites were fixed on neutral pH paper as the base instead.

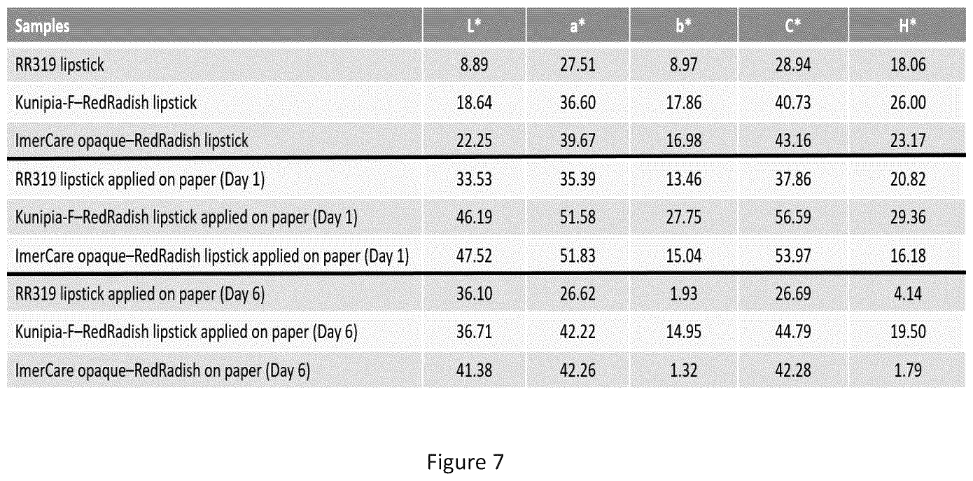

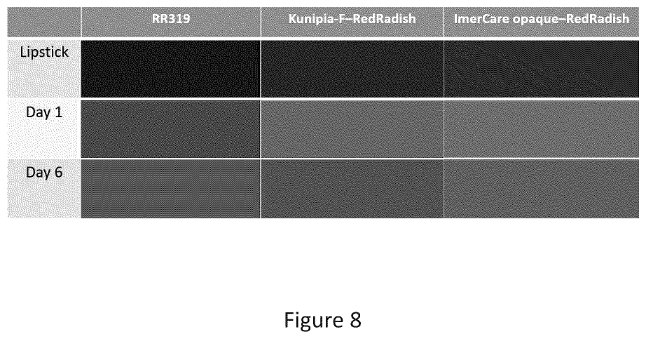

The pigment composites Kunipia-F-RR and ImerCare Opaque-RR were applied onto the paper and the L*, a*, b*, C* and h values were measured immediately using the X-rite spectrophotometer which was subsequently labelled as Day 1. The last measurement was taken 6 days later and recorded in a table shown in FIG. 7 with corresponding measured color in FIG. 8.

It is a known phenomenon that the natural red radish extract does experience blue-shifting in color as it is influenced by the pH-dependent anthocyanins. Based on the results shown in FIGS. 7 and 8, the Kunipia-F-RR and ImerCare opaque-RR pigment composites in lipsticks are physically brighter and redder as compared to the Xfine RR319 lipstick. As for the performance, on being applied on neutral pH paper, the freshly applied Kunipia-F-RR pigment composite exhibited the best result, showing physically the brightest and reddest (tint of orange) on the neutral pH paper. With the observation progressing towards day 6, it is notable that the red shade of Xfine RR319 and ImerCare opaque-RR both have turned purple at the end of day 1 while the red shade of Kunipia-F-RR remains stable. At the end of day 6, only the Kunipia-F-RR maintained its red shade with highest a* and b* while the rest of the two have turned purplish and pinkish, respectively.

The Kunipia-F-RR pigment composite managed to exhibit good stability on the bentonite with the pH-dependent anthocyanins beyond day 1 and well maintained with progression towards day 6. This behavior is attributed the physiochemical properties of the bentonite having electrostatic interaction attraction with the anthocyanins in the extract.

With the concluded investigation on Raphanus sativus L., the sodium bentonite (Kunipia-F) from Kunimine Industries and kaolin (ImerCare opaque) from Imerys were deployed for the rest of the case studies on various vegetable/fruit extracts.

### Example 6: Another Source of Anthocyanins from Ipomoea batatas L. [Purple Sweet Potato]

The second case study of the present invention provides a bluish-red to pink shade using Ipomoea batatas L. extract powder as the source of anthocyanins. The source of Ipomoea batatas L extract powder was obtained from commercially attainable products: “Purple Sweet Potato (PSP) Extract” from Sensient Beauty.

In this preferred embodiment, the pigment composite comprises from about 5 wt % to about 30 wt % of Purple Sweet Potato Extract, from about 25 wt % to about 40 wt % of bentonite (Kunipia-F), from about 30 wt % to about 45 wt % of maltodextrin and from about 8 wt % to about 15 wt % of citric acid. The citric acid is added to serve as pH adjustment ranging from 2.9 to 3.4 but preferably at 3.0.

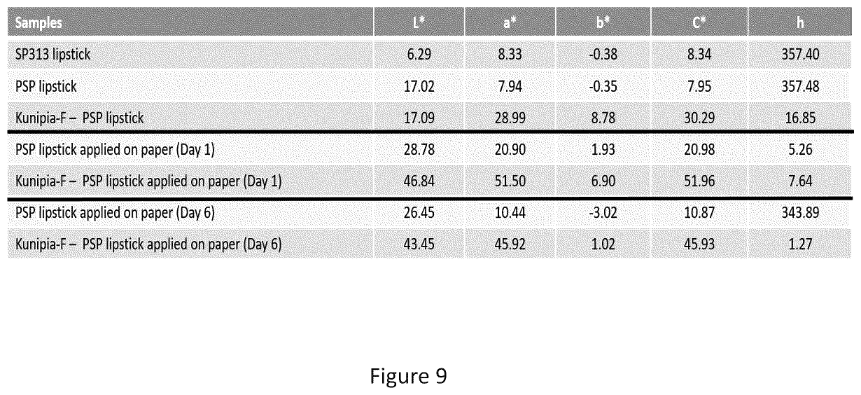

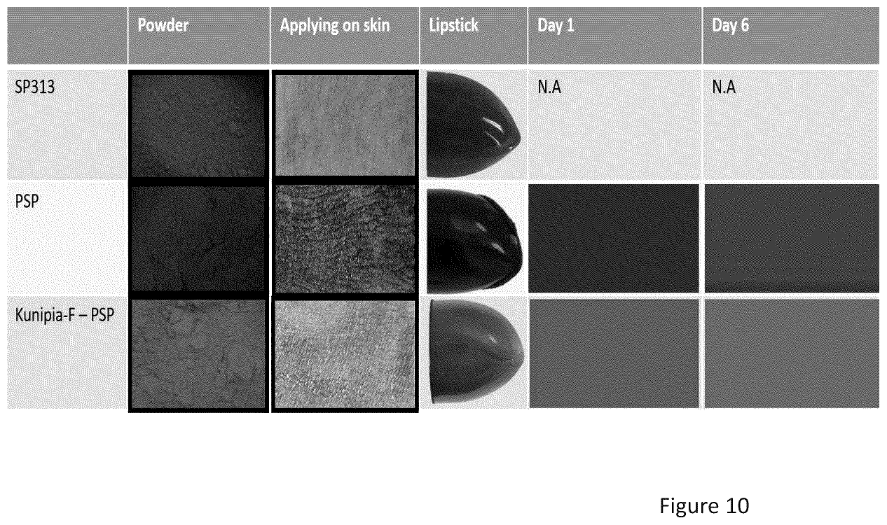

The Natpure Xfine Potato SP313 is used as an example for illustration purposes but it is not included in the colorimetric analysis due to low color strength and poor coverage in lipstick application. FIG. 9) records the colorimetric values measured by the spectrophotometer while FIG. 10 depicts the physical appearance of the powder and lipsticks after applied onto the skin.

Based on the investigation conducted in the first case study, the color evolution and evaluation will be measured on the day 1 and on the day 6 for this new Kunipia-F-PSP versus the raw material PSP study. FIGS. 9 and 10 have distinctively illustrated and proven that the bentonite (Kunipia-F) could stabilize and maintain the anthocyanins in the purple sweet potato extract. The electronic data shown in FIG. 9 indicates that there is slight blue-shifting occurring on the Kunipia-F-PSP pigment composite but minimal changes on its brightness and redness. The raw material PSP is chosen as a benchmark to highlight the stabilizing effect of the bentonite on the anthocyanins.

The Ipomoea batatas L. investigation has further supported the conclusion stated in the first case study using the raphanus sativus L. extract. This investigation has proven that the deployment of the bentonite (as Kunipia-F) can be extended to other natural extract containing anthocyanins regardless of which type of anthocyanins predominate in the extract.

### Example 7: Non-Anthocyanin Source as Natural Dye-Genipin-hydrolyzed Gardenia jasminoides ellis [Gardenia Blue]

This case study of the present disclosure provides a blue shade from non-anthocyanins extract such as the Gardenia jasminoides ellis extract. The source of Gardenia jasminoides ellis extract powder was obtained from commercially attainable products: “Natpure Xfine Gardenia GB618” from Sensient Beauty.

In this preferred embodiment, the pigment composite comprises from about 1 wt % to about 15 wt % of Xfine Gardenia GB618 powder, from about 25 wt % to about 40 wt % of bentonite (Kunipia-F), and from about 60 wt % to about 75 wt % of maltodextrin.

In another preferred embodiment, the pigment composite comprises from about 1 wt % to about 15 wt % of Xfine Gardenia GB618 powder, from about 40 wt % to about 55 wt % of kaolin (ImerCare Opaque), and from about 40 wt % to about 55 wt % of maltodextrin.

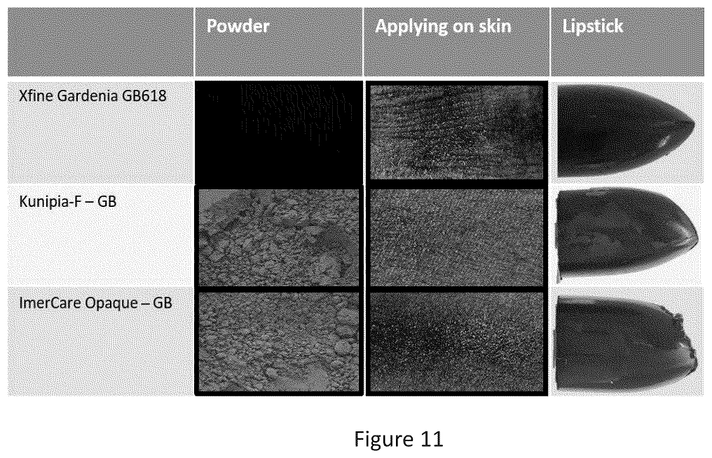

Based on physical observation shown in FIG. 11, the investigation on this non-anthocyanin case study definitively demonstrates that the bentonite (Kunipia-F) gives the warmer shade (yellowish tone) of the pigment composite while the kaolin (ImerCare Opaque) produces a cooler shade (bluish tone). This study demonstrates that the bentonite (Kunipia-F) generally is not only able to provide brighter shade (yellowish tone) to natural extract that contain anthocyanins but can be also extended to other natural extract containing genipin-amino acid complex pigment (Gardenia Blue or Jagua Blue). The same behavior was also observed on the kaolin (ImerCare opaque) as a co-substrate providing brighter shade (bluish tone) with underlying high brightness reflectance index, regardless of whether the natural extract contains anthocyanins or amino-acid complex extract.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising:
a natural dye in an amount by weight of between 5% and 35%;
a bentonite clay substrate in an amount by weight of between 45% and 60%;
maltodextrin in an amount by weight of between 10% and 45%; and
an optional pH adjuster,
wherein the natural dye is at least partly integrated into a cation interlayer of the bentonite clay substrate by way of a spray drying process,
wherein the composition maintains color stability after storage at 28° C, and 68% relative humidity for 30 days,
wherein color stability includes maintaining at least 50% of one or more of an L* value, an a* value, or a b* value, as measured by reflectance visible spectroscopy at one or more wavelengths between 400 nm and 800 nm,
wherein over 50% of cations within the cation interlayer are sodium cations,
wherein the bentonite clay substrate has, prior to having the natural dye at least partly integrated into the cation interlayer, a whiteness of at least 50%, and
wherein the bentonite clay substrate has, prior to having the natural dye at least partly integrated into the cation interlayer, an initial cation exchange capacity of at least 75 meq/100 g.

2. A composition comprising:
a natural dye in an amount by weight of between 20% and 35%;
a kaolin clay substrate in an amount by weight of between 55% and 70%;
an optional pH adjuster; and
an optional surface treatment,
wherein the composition maintains color stability after storage at 28° C, and 68% relative humidity for 30 days,
wherein color stability includes maintaining at least 50% of one or more of an L* value, an a* value, or a b* value, as measured by reflectance visible spectroscopy at one or more wavelengths between 400 nm and 800 nm, and
wherein the kaolin clay substrate, prior to contacting the natural dye, has a reflectance brightness of at least 85% as measured by visible reflectance spectroscopy.

3. The composition of claim 2, the composition comprising the surface treatment, wherein the surface treatment is selected from the group consisting of a stearate, optionally polyglyceryl-10-pentaisostearate or polyglyceryl-10 nonaisostearate, a wax, optionally Meadowfoam wax, an oil or butter, optionally Moringa oil or butter, optionally aloe oil or butter, silicone, and combinations thereof.

4. The composition of claim 1, wherein the natural dye has color that varies with pH, the composition comprising the pH adjuster.

5. The composition of claim 4, wherein the pH adjuster is citric acid.

6. The composition of claim 4, wherein the pH adjuster is present in the composition in an amount by weight of between 8% and 15%.

7. The composition of claim 1 further comprising a co-substrate, wherein the co-substrate comprises a polysaccharide.

8. A filled composition comprising the composition of claim 1, further comprising at least one filler, wherein the filler comprises mica, talc, silica, or calcium carbonate.

9. The composition of claim 1, further comprising at least one mineral pigment, the mineral pigment comprising boron nitride, or a clay.

10. The composition of claim 1, wherein the natural dye is an anthocyanin, a genipin-amino acid complex, or a combination thereof.

11. A method of preparing a powder composition, comprising the following steps:
a) homogenizing a natural dye with a solvent to form a slurry with either: i) a bentonite clay having a whiteness of at least 50% and a cation exchange capacity (CEC) of at least 75 meq/100 g; or ii) a kaolin clay with a reflectance brightness of at least 85%,
b) optionally adjusting pH of the slurry by adding a pH adjuster, and
c) spray-drying the slurry with an outlet temperature between 60° C, and 120° C. to form a powder.

12. The method of claim 11, further comprising step d) performing a particle size reduction process to reduce a particle size of the powder composition to between 1 μm and 20 μm, wherein the particle size reduction process comprises milling, grinding, pulverizing, or a combination thereof.

13. The method of claim 11 wherein the solvent is water, a volatile organic solvent, or an emulsion base.

14. The method of claim 11 wherein a particle size of the powder composition is between 2 μm and 10 μm.

15. The method of claim 11 wherein a moisture content is between 0 wt % and 10 wt %.

16. The method of claim 11 further comprising a step (v) combining particles of the powder composition with an additive, wherein the additive includes an oil, a butter, or a wax.

17. The method of claim 16 wherein the additive comprises polyglyceryl-10 pentaisostearate, meadowfoam wax, polyglyceryl-10 nonaisostearate, moringa oil, moringa butter, aloe oil, aloe butter, and combinations thereof.

18. The method of claim 11 wherein the pH adjuster is an acid, the acid comprising citric acid, ascorbic acid, an alpha hydroxy acid, a beta hydroxy acid, a volatile acid, or a fatty acid, or a combination thereof.

19. The method of claim 11 wherein the pH adjuster is a base, the base comprising amines, organic amines, ammonium hydroxide, sodium hydroxide, potassium hydroxide, or a combination thereof.

20. (canceled)

21. A natural pigment composition comprising:
a natural dye in an amount by weight of between 3% and 40%;
a clay substrate in an amount by weight of between 20% and 75%;
an optional co-substrate in an amount by weight of between 0% and 45%;
an optional pH adjuster; and
an optional surface treatment in an amount by weight of between 1% and 15%,
wherein the natural pigment composition is in powder form.

22-41. (canceled)
